(19) United States
(12) Patent Application Publication
(10) Pub. No.: US 2026/0041482 A1
(43) Pub. Date: Feb. 12, 2026

(54) HEMORRHOID TREATMENT SYSTEM AND ASSOCIATED METHODS

(71) Applicant: Midfiniti International, Inc., Las Vegas, NV (US)

(72) Inventors: Stephen Goldman, Palm Harbor, FL (US); Michael J. Goeree, Tampa (CA)

(21) Appl. No.: 18/795,908

(22) Filed: Aug. 6, 2024

(51) Int. Cl. A61B 18/14; A61B 17/00; A61B 18/00; A61B 18/16; A61B 90/30; G16H 10/60; G16H 15/00

(52) U.S. Cl. CPC A61B 18/1485; A61B 18/16; A61B 90/30; G16H 10/60; G16H 15/00; A61B 2017/00199; A61B 2018/005; A61B 2018/00648; A61B 2018/00755; A61B 2018/00827; A61B 2018/00892; A61B 2018/167

(57) ABSTRACT

A hemorrhoid treatment system includes a computing device, and a handheld device coupled to the computing device. The handheld device includes a probe electrode to contact a hemorrhoid on a patient that is to be treated. A grounding pad is to be coupled to the patient. The handheld device includes a variable resistance network, and a controller to sense a feedback voltage at a current sensing resistor in response to the probe electrode contacting the hemorrhoid and to the patient being grounded via the grounding pad. A level of current being delivered to the hemorrhoid is determined based on the sensed feedback voltage. Resistance of the variable resistance network is changed to adjust the output voltage to compensate for fluctuations in body resistance of the patient so that the level of current being delivered to the hemorrhoid matches a set level of current.

### TECHNICAL FIELD

The present disclosure relates to the treatment of hemorrhoids, and, more particularly, to a hemorrhoid treatment system that delivers an electrical current to a hemorrhoid that is to be treated, and associated methods.

### BACKGROUND

Hemorrhoids, also called piles, are swollen veins in the anus and lower rectum. As the hemorrhoidal vein becomes more dilated from the increased pressure, it may eventually form a sac-like protrusion if the condition is allowed to persist. Hemorrhoids are generally classified as being either internal or external, depending on their location relative to the dentate line.

The pain often associated with hemorrhoids may be great enough to interfere with normal patterns of defecation which in turn leads to constipation and further aggravation of the swelling in the region. In general, most symptoms associated with hemorrhoids are sufficient enough to interfere with a person's daily life causing them to require relief from a medical practitioner and/or a pharmaceutical preparation.

There is a wide range of therapies available that attempt to provide a person suffering from hemorrhoids with some level of relief. One option is medicated ointments and creams which generally relieve either the itching or inflammation, but few have been successful in reducing or completely eliminating both. Thus, the efficacy of medicated ointments and creams in relieving or curing the symptoms of hemorrhoids is uncertain.

Another option for treating hemorrhoids is surgery. Treatment via surgery ranges from minor surgery to major invasive surgery. Minor surgery may relieve symptoms through drainage of the swelling. Major invasive surgery, for example, includes the removal of extensive or severe hemorrhoids in a process known as a hemorrhoidectomy. This surgical procedure can be used on both internal and external hemorrhoids. However, a hemorrhoidectomy typically involves a long recovery period, along with the associated risks and expense of invasive surgery.

Another example for treating hemorrhoids is to deliver an electrical current to the hemorrhoids. U.S. Pat. No. 9,179,966 provides a method of treating hemorrhoids that includes inputting a predetermined current time unit into a current delivering medical instrument, wherein the predetermined current time unit is based on the grade of the hemorrhoid. The hemorrhoid is contacted with a probe electrode of the current delivering medical instrument, and current is delivered to the hemorrhoid. A delivered current time unit is determined. The delivered current time unit includes a product comprised of current delivered multiplied by time. A visual and/or auditory indication is provided with the medical instrument when the predetermined current time unit amount is achieved.

Nonetheless, even in view of the above, there is still a need to improve how hemorrhoids are treated.

### SUMMARY

A hemorrhoid treatment system includes a computing device and a handheld device. The handheld device has a cable coupled to the computing device, with the cable comprising an inline connector junction. A probe electrode may have a first end coupled to the handheld device, and a second end to contact a hemorrhoid on a patient that is to be treated. A grounding pad may be coupled to the patient. A grounding pad cable may be coupled between the grounding pad and the inline connector junction.

The handheld device may include a voltage output configured to deliver an output voltage to the probe electrode, a variable resistance network coupled to the voltage output, a current sensing resistor configured to provide a feedback voltage, and a controller coupled to the current sensing resistor and the variable resistance network.

The controller may be configured to sense the feedback voltage at the current sensing resistor in response to the second end of the probe electrode contacting the hemorrhoid and to the patient being grounded via the grounding pad, and determine a level of current being delivered to the hemorrhoid based on the sensed feedback voltage. The controller may be configured to change resistance of the variable resistance network to adjust the output voltage to compensate for fluctuations in body resistance of the patient so that the level of current being delivered to the hemorrhoid matches a set level of current.

The controller may periodically sense the feedback voltage at the current sensing resistor. The variable resistance network may include a plurality of resistive inputs, with each resistive input corresponding to a different resistive value. The controller may include a sensing and adjusting algorithm to periodically sense the feedback voltage, and to adjust the resistance of the variable resistance network by selecting one of the plurality of resistive inputs so that the level of current being delivered to the hemorrhoid matches the set level of current.

The cable may be configured to provide power and ground from the computing device to the handheld device. The grounding pad cable may be coupled to ground in the inline connector junction.

The computing device may include operational treatment software configured to perform handshaking with the handheld device in order for the handheld device to operate. The operational treatment software may also be configured to generate a treatment summary report in response to the handheld device being powered off after treatment of the hemorrhoid.

The treatment summary report may be stored in a medical records database. The treatment summary report may include identification of the patient, a duration of how long current was delivered to the hemorrhoid, and the level of the current delivered to the hemorrhoid during the duration.

The handheld device may include a timer display, and may be configured to operate in a manual mode or a timer mode. The manual mode may correspond to the timer display counting up from zero during treatment of the hemorrhoid. The timer mode may correspond to the timer display counting down from a selected time set on the timer display.

The handheld device may include a current bar graph display to display the current level being delivered to the hemorrhoid.

The probe electrode may be bifurcated as a pair of spaced apart elongated electrode members each with an electrode tip for contacting the hemorrhoid.

A spacer may slidably engage the elongated electrode members in order to maintain a desired spacing between the electrode tips.

A base overmold may be positioned over a portion of the elongated electrode members, and an open extension may extend from the base between inward facing surfaces of the elongated electrode members while exposing outward facing surfaces of the elongated electrode members.

The probe electrode may be a continuous length of conductive material folded in half to define a closed end and an open end. The closed end may be configured as a spring for press-fitting into the handheld device. The open end may be configured as a pair of spaced apart electrode tips for contacting the hemorrhoid.

The handheld device may include a housing enclosing the voltage output, the variable resistance network, the current sensing resistor and the controller. A plastic insert may be secured within the housing and has an opening extending therethrough. An electrical plug may have a receptacle end and a threaded end, with the receptacle end positioned within the opening in the plastic insert and configured to receive the first end of the probe electrode. The threaded end may extend through a backside of the plastic insert. An electrical lug may be positioned over the threaded end of the electrical plug for contacting the voltage output. A nut may be secured to the threaded end of the electrical plug for securing the electrical plug within the plastic insert.

The handheld device may include at least one light directed to the second end of the probe electrode that contacts the hemorrhoid.

Another aspect is directed to a handheld device that may include a housing, an electrical plug carried by the housing and having a receptacle end configured to receive a first end of a probe electrode, with a second end of the probe electrode configured to contact a hemorrhoid on a patient that is to be treated. A voltage output may be carried by the housing and configured to deliver an output voltage to the probe electrode via the electrical plug. A variable resistance network may be carried by the housing and coupled to the voltage output. A current sensing resistor may be carried by the housing and configured to provide a feedback voltage. A controller may be carried by the housing and coupled to the current sensing resistor and the variable resistance network. The controller may be configured to sense the feedback voltage at the current sensing resistor in response to the second end of the probe electrode contacting the hemorrhoid and to the patient being grounded, and determine a level of current being delivered to the hemorrhoid based on the sensed feedback voltage. The controller may change resistance of the variable resistance network to adjust the output voltage to compensate for fluctuations in body resistance of the patient so that the level of current being delivered to the hemorrhoid matches a set level of current.

Yet another aspect is directed to a method for using a handheld device to treat a hemorrhoid on a patient as described above. The method may include grounding the patient via a grounding pad, providing power to the handheld device, and positioning the handheld device so that a second end of the probe electrode contacts the hemorrhoid on the patient. The method may include operating the controller to sense the feedback voltage at the current sensing resistor in response to the second end of the probe electrode contacting a hemorrhoid on a patient that is to be treated and to the patient being grounded, and determine a level of current being delivered to the hemorrhoid based on the sensed feedback voltage. The controller may change resistance of the variable resistance network to adjust the output voltage to compensate for fluctuations in body resistance of the patient so that the level of current being delivered to the hemorrhoid matches a set level of current.

### BRIEF DESCRIPTION OF THE DRAWINGS

FIG. 1 is a block diagram of a hemorrhoid treatment system in which various aspects of the disclosure may be implemented.

FIG. 2 is a view of the handheld device, probe electrode, grounding pad and grounding pad cable illustrated in FIG. 1.

FIG. 3 is a back side perspective view of the handheld device and probe electrode illustrated in FIG. 1 without the cable.

FIG. 4 is a front side perspective view of the handheld device and probe electrode illustrated in FIG. 1 without the cable.

FIG. 5A is a side view of a single length of electrically conductive material bent in half to form part of the probe electrode illustrated in FIG. 1.

FIG. 5B is a side view of the electrically conductive material illustrated in FIG. 5A with shrink tubing.

FIG. 5C is a back side perspective view of the electrically conductive material with shrink tubing illustrated in FIG. 5B with a base overmold.

FIG. 6A is a back side perspective view of the probe electrode illustrated in FIG. 5C with a slidable spacer.

FIG. 6B is a front view of the slidable spacer illustrated in FIG. 6A.

FIGS. 7A-7C are different views of another embodiment of the probe electrode illustrated in FIG. 5C with a base overmold closed extension.

FIGS. 8A-8C are different views of another embodiment of the probe electrode illustrated in FIG. 5C with a base overmold open extension.

FIG. 9 is a partially exploded view of the handheld device configured to receive the probe electrode illustrated in FIG. 1.

FIG. 10 is a partial view of the probe electrode coupled to the handheld device illustrated in FIG. 9.

FIGS. 11A-11C are sequence views of the closed end of the probe electrode being inserted into the electrical plug within the handheld device illustrated in FIG. 10.

FIG. 12 is a view of a user interface membrane placed on the user interface area of the handheld device illustrated in FIG. 3.

FIG. 13 is a flowchart for using the handheld device illustrated in FIG. 1 to treat a hemorrhoid on a patient.

### DETAILED DESCRIPTION

The present description is made with reference to the accompanying drawings, in which exemplary embodiments are shown. However, many different embodiments may be used, and thus the description should not be construed as limited to the particular embodiments set forth herein. Rather, these embodiments are provided so that this disclosure will be thorough and complete. Like numbers refer to like elements throughout.

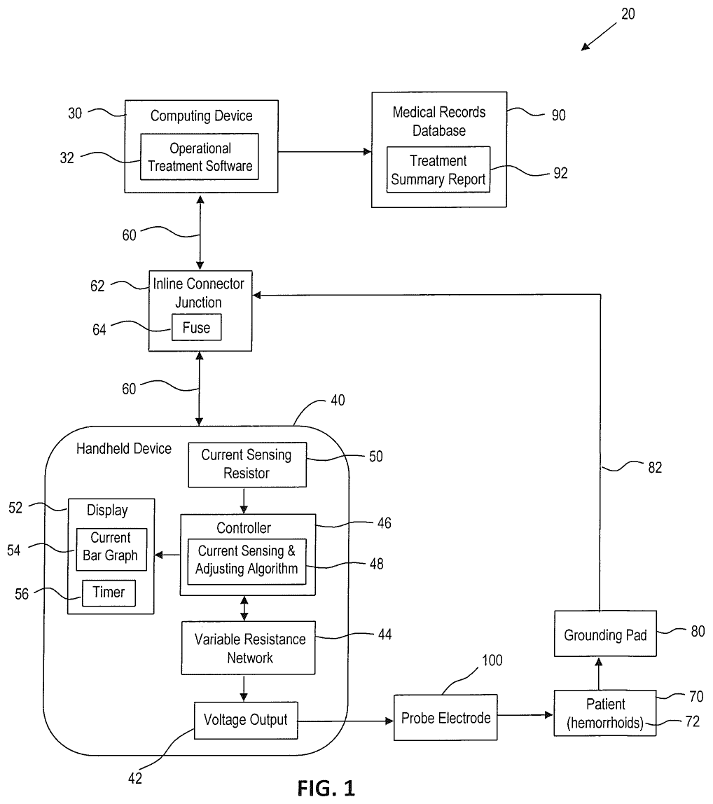

Referring initially to FIG. 1, a hemorrhoid treatment system 20 for a patient 70 having hemorrhoids 72 that are to be treated will be discussed. The hemorrhoid treatment system 20 utilizes direct current electrotherapy to reduce and eliminate symptoms of hemorrhoid disease, which is estimated to affect up to one-third of the population in the United States.

As will be described in detail below, the hemorrhoid treatment system 20 is advantageously configured to automatically deliver a steady level of current to the hemorrhoid 72 by using feedback to adjust an output voltage to compensate for fluctuations in body resistance of the patient 70. As noted in the above Background section, U.S. Pat. No. 9,179,966 disclosed the use of electrical current to treat hemorrhoids. A limitation of the '966 patent is that manual intervention was required to adjust for variations in the level of current being delivered to the hemorrhoid due to fluctuations in body resistance of the patient.

The hemorrhoid treatment system 20 includes a computing device 30, and a handheld device 40 coupled to the computing device 30 via a cable 60. The computing device 30 is configured to provide power and ground to the handheld device 40 over the cable 60, as well as to exchange data signals with the handheld device 40.

The computing device 30 may be a laptop or desktop computing device, for example. The computing device 30 is configured to execute operational treatment software 32 for interfacing with the handheld device 40 over the cable 60. One of the functions of the operational treatment software 32 is to perform handshaking with the handheld device 40. Handshaking is required to authorize operation of the handheld device 40. After authorization, operation of the handheld device 40 may be self-sustained to operate on its own.

The operational treatment software 32 is also configured to generate a treatment summary report 92 after the patient 70 has been treated, which may be used for billing purposes. The treatment summary report 92 may include identification of the patient, a duration of how long current was delivered to the hemorrhoid 72, and the level of the current delivered to the hemorrhoid 72 during the duration. The patient identification may be, for example, by name, social security number, or an assigned patient number. The treatment summary report 92 may be stored in a medical records database 90.

A probe electrode 100 has a first end coupled to the handheld device 40, and a second end configured to contact or engage the hemorrhoid 72 that is to be treated. A grounding pad 80 is to be coupled to the patient 70. The cable 60 includes an inline connector junction 62. A grounding pad cable 82 is coupled between the grounding pad 80 and the inline connector junction 62, where the inline connector junction 62 provides a ground to the grounding pad 80. The inline connector junction 62 may include a fuse 64 in series between the grounding pad 80 and a grounding wire within the cable 60 to limit a maximum amount of current that may be delivered to the hemorrhoid 72.

The handheld device 40 includes a voltage output 42 configured to deliver an output voltage to the probe electrode 100, a variable resistance network 44 coupled to the voltage output 42, and a current sensing resistor 50 configured to provide a feedback voltage. The variable resistance network 44 has a plurality of selectable resistance configurations.

A controller 46 is coupled to the current sensing resistor 50 and to the variable resistance network 44. The controller 46 is configured to operate a current sensing and adjusting algorithm 48 to periodically sense the feedback voltage at the current sensing resistor 50 in response to the second end of the probe electrode 100 contacting the hemorrhoid 72, and to the patient 70 being grounded via the grounding pad 80. The patient 70 acts as a resistive load to form an electrical circuit between the probe electrode 100 and the grounding pad 80.

A level of current being delivered to the hemorrhoid 72 is determined by the current sensing and adjusting algorithm 48 using Ohm's law. Ohm's law is a formula used to calculate the relationship between voltage, current and resistance in an electrical circuit.

In response to the probe electrode 100 contacting the hemorrhoid 72 and the patient 70 being grounded via the grounding pad 80, an output voltage is applied to the probe electrode 100. The current flowing through the hemorrhoid 72 ramps up from 0 to 16 milliamps (mA) within about 10 seconds. The electrical current is a steady, low-level current that causes smooth muscle contraction and thrombosis resulting in permanent ligation of the hemorrhoid's feeder blood vessels. The use of 16 mA is one example current level that may be used for treating hemorrhoids 72. A 16 mA current may be applied for a time determined by the hemorrhoid's grade for a 90-95% cure rate. The doctor or gastroenterologist will determine the hemorrhoid grade and thus choose the procedural time needed. Since the vast majority of patients are unable to tolerate 16 mA of current, anesthesia is typically used.

As noted above, the level of current being delivered to the hemorrhoid 72 is determined by the controller 46 based on the feedback voltage being measured at the current sensing resistor 50. The feedback voltage is periodically measured. As an example, this measurement may be in the milliseconds range, such as every 32 milliseconds.

The handheld device 40 includes a display 52 coupled to the controller 46. The display 52 provides a current bar graph 54 and a timer 56. The current bar graph 54 displays the level of current being delivered to the hemorrhoid 72. When current is being delivered to the hemorrhoid 72, the timer 56 changes time. The handheld device 40 may operate in a manual mode or a timer mode. In the manual mode, the timer 56 counts up from zero. In the timer moder, the timer 56 counts down to zero from a time selected by the doctor.

In response to the level of current being delivered to the hemorrhoid 72, the controller 46 changes resistance of the variable resistance network 44 to adjust the output voltage to compensate for fluctuations in body resistance of the patient 70 so that the level of current being delivered to the hemorrhoid 72 matches a desired or set level of current. The desired level of current is 16 mA in this example embodiment, and may be considered a default value for the level of current to be applied to the hemorrhoid 72 without additional input from the doctor.

The controller 46 may have a plurality of output ports that interfaces with a corresponding plurality of inputs to the resistive network 44, where each input corresponds to a different resistance configuration. The controller 46 46 changes resistance of the variable resistance network 44 by selecting the appropriate output port that connects to a desired resistance configuration within the resistive network 44.

Fluctuations in body resistance, for example, may be due to movement by the patient 70 with respect to the grounding pad 80. In addition, saline is a conductive solution that is typically used to apply the grounding pad 80 to the patient 70. If the saline starts to dry out, then this may lead to fluctuations in body resistance of the patient 70. The handheld device 40 advantageously treats the hemorrhoid 72 by delivering the desired level of current to the hemorrhoid 72 within a very tight tolerance. The tolerance may be within a range of 0.5-1.5 percent, for example.

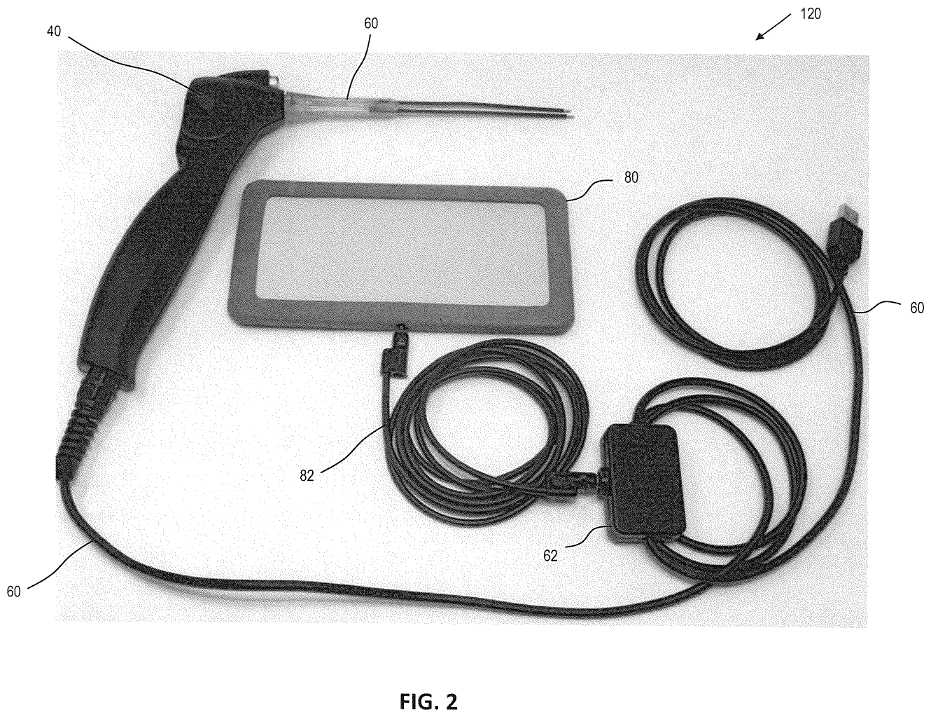

A view 120 of the handheld device 40 with cable 60 and inline connector junction 62, probe electrode 100, grounding pad 80 and grounding pad cable 82 is provided in FIG. 2. Since the grounding pad 80 is disposable, it is removably coupled to one end of the ground pad cable 82. The other end of the grounding pad cable 82 is removably coupled to the inline connector junction 62. One end of the cable 60 is coupled within the handheld device 40, with the free end of the cable 60 being removably coupled to the computing device 30. The cable 60 may be configured as a USB (universal serial bus) cable. A USB cable allows data exchanges between the computing device 30 and the handheld device 40, and provides power and ground to the handheld device 40 as well as providing ground to the ground pad 80 via the inline connector junction 62.

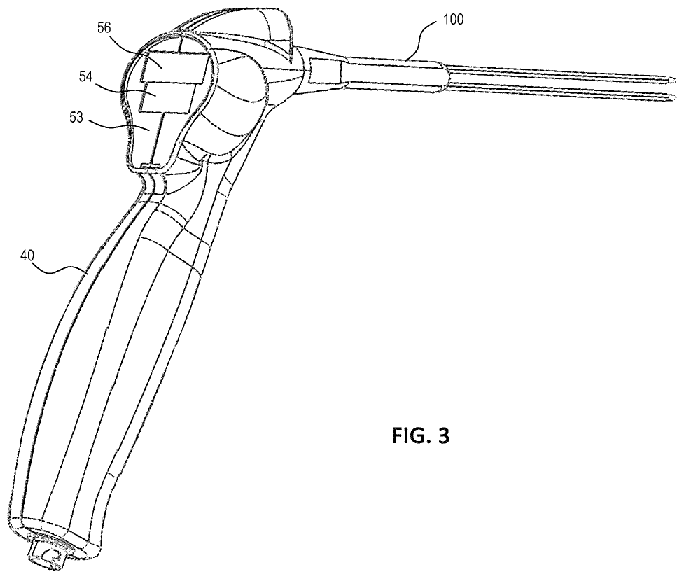

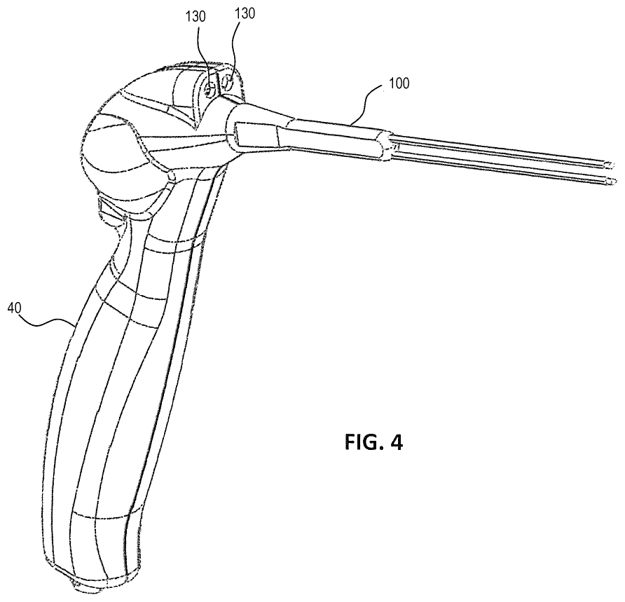

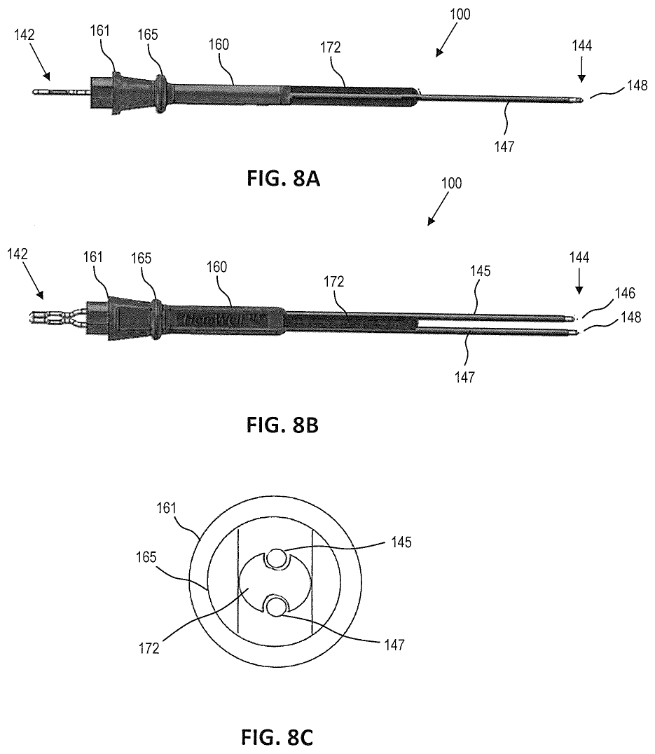

Referring now to FIGS. 3 and 4, a back side perspective view and a front perspective view of the handheld device 40 with the probe electrode 100 are provided. The handheld device 40 includes a user interface area 53 that is visible to the doctor from the backside of the handheld device 40. The display area 53 includes the current bar graph 54 and the timer 56. As will be discussed in greater detail below with reference to FIG. 9, a user interface membrane 190 is placed over the display area 53 which provides user interface buttons for operating the handheld device 40.

The handheld device 40 further includes at least one light 130 directed to the second end of the probe electrode 100 that contacts the hemorrhoid. This results in safer and more accurate procedures. In the illustrated embodiment, a pair of lights 130 are provided. The lights may be configured as light emitting diodes (LEDs).

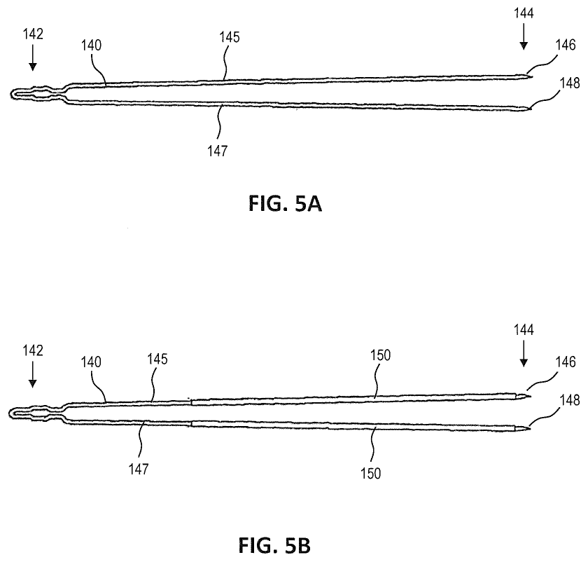

Referring now to FIGS. 5A-5C, the probe electrode 100 will be discussed in greater detail. The probe electrode 100 includes a single length of electrically conductive material 140 that is folded in half, with a bend region forming a closed end 142 of the probe electrode 100, as shown in FIG. 5A. Extending from the bend region is a first elongated electrode member 145 that includes a first electrode tip 146, and a second elongated electrode member 147 that includes a second electrode tip 146. The first and second electrode tips 146, 148 form an open end of the probe electrode 100. Each electrode tip 146, 148 is preferably beveled to form a point.

The closed end 142 corresponds to the first end of the probe electrode 100, and the open end 144 corresponds to the second end of the probe electrode 100. In other words, the probe electrode 100 is bifurcated as a pair of spaced apart elongated electrode members 145, 147 each with an electrode tip 146, 148 for contacting the hemorrhoid 72.

The electrically conductive material may be a metal wire, such as stainless steel, copper, etc. The metal wire is heat annealed at approximately its center point, and is then bent about 180 degrees to form the bend region. This allows for the electrode tips 146, 148 to be divergent from one another.

Shrink tubing 150 is placed over the first elongated electrode member 145 and over the second elongated electrode member 147 while leaving the respective electrode tips 146, 148 exposed, as shown in FIG. 5B. The single length of electrically conductive material 140 with the shrink tubing 150 thereon is placed into an oven at a temperature and time sufficient to shrink the tubing 150 around the respective first and second elongated electrode members 145, 147. Typically, 15 minutes @ 300-500 degrees F. is sufficient for this purpose.

After shrinking the tubing 150, a base overmold 160 is associated with the first and second elongated electrode members 145, 147, as shown in FIG. 5C. The base overmold 160 provides a grip area for holding the probe electrode 100 while interfacing (i.e., coupling/decoupling) with the handheld device 40. In one embodiment this is achieved through plastic mold injection. The molds are designed to seal around a portion of the first and second elongated electrode members 145, 147 including a portion of the shrink tubing 150.

In one embodiment, the plastic used for the base overmold 160 is C10 made by Adept Polymers, Ltd, and the injection is conducted @ 11,000 PSI, @ 350-400 degrees F. The plastic injection forms a 4-sided keying block 162 with a physical stop 163. This is intended for mating with the handheld device 40. The 4-sided keying block 162 allows for the positioning of the probe electrode 100 in two vertical positions and two horizontal positions.

As noted above, the elongated electrode members 145, 147 and the electrode tips 146, 148 are divergent from one another. A default spacing of the electrode tips 146, 148 is obtained when the base overmold 160 is formed. For a default spacing of 5 mm, for example, the electrode tips 146, 148 are held at this spacing while the base overmold 160 is formed. After the base overmold 160 has been formed, the default spacing of the electrode tips 146, 148 is set. The default spacing of the electrode tips 146, 148 is not limited to 5 mm. The default spacing may vary within a range of 5 mm-12 mm, for example.

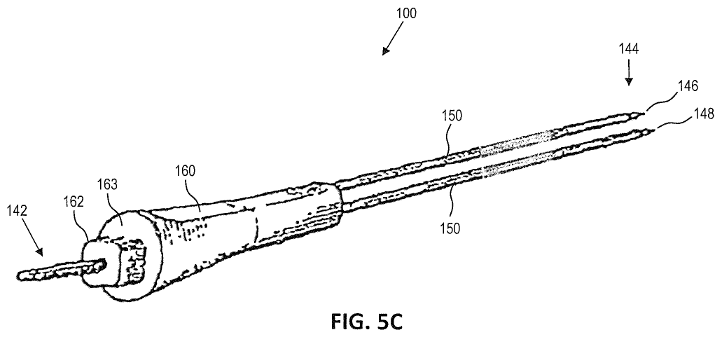

To ensure that spacing of the electrode tips 146, 148 is at the default range, or if the doctor would like to change the default spacing, a spacer 170 may be used, as shown in FIG. 6AC. The spacer 170 includes a pair of spaced apart openings 172, as shown in FIG. 6B, for slidably engaging the elongated electrode members 145, 147 of the probe electrode 100.

The spacer 170 may be formed out of an elastic, flexible and moldable material, such as polyurethane or rubber, for example. Prior to treating the hemorrhoid 72, the doctor slides the spacer 170 over the electrode tips 146 148 and over the elongated electrode members 145, 147 until the desired spacing is achieved. The shrink tubing 150 placed over the elongated electrode members 145, 147 may include one or more markers calibrated to assist in positioning of the spacer 170 to achieve the desired spacing.

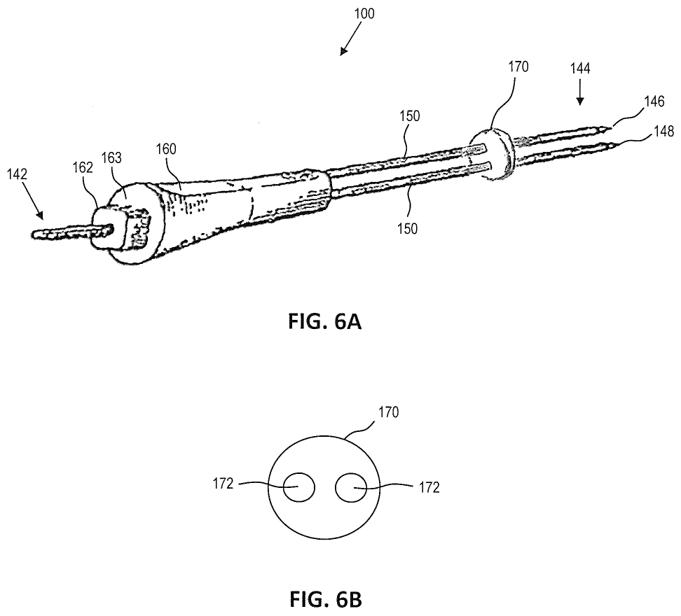

Referring now to FIGS. 7A-7C, the base overmold 160 may be formed with a closed extension 170. The closed extension 170 is a continuation of the base overmold 160 and partially extends over the elongated electrode members 145, 147. The closed extension 170 helps to insure that the electrode tips 146 148 are held at the default spacing.

The base overmold 160 may be formed with base sections 161, 165 such that a portion of the base overmold 160 therebetween is tapered from base section 161 towards base section 165. From base section 165 towards the electrode tips 146, 148, a profile of the base overmold 160 matches a profile of the closed extension 170.

Referring now to FIGS. SA-8C, the base overmold 160 may be formed with an open extension 172. The open extension 172 is a continuation of the base overmold 160 but exposes the outward facing surfaces of the elongated electrode members 145, 147. The open extension 172 is formed between the inward facing surfaces of the elongated electrode members 145, 147. A profile of the open extension 172 is circular and includes a pair of openings to receive the inward facing surfaces of the elongated electrode members 145, 147 while the outward facing surfaces are exposed.

Although not shown, the final packaging of the probe electrode 100 includes a plastic guard to prevent accidental puncture and a gas permeable sleeve for sterilization. Due to the plastic being water-soluble, gas sterilization or E-Beam is used. Steam sterilization is prohibited.

Referring now to FIG. 6, a partially exploded view of the handheld device 40 configured to receive the probe electrode 100 will be discussed. The handheld device 40 includes a housing 41 that defines a shape of the handheld device 40. The housing 41 may be formed by joining together two housing halves. One of the housing halves 41 is removed to illustrate where the first end of the probe electrode 100 is to be coupled to the handheld device 40.

A plastic insert 180 is secured within the housing 41. The housing 41 includes a tapered extension and slotted fins within the housing. The plastic insert 180 is held in place by being positioned between the tapered extension and the slotted fins. The plastic insert 180 has an opening extending therethrough, and an electrical plug 182 is to be positioned within the opening. The electrical plug 182 has a receptacle end 183 and a threaded end 185. The receptacle end 183 is configured to receive the closed end 142 of the probe electrode 100. The threaded end 183 is to extend through a backside of the plastic insert 180.

An electrical lug 184 is to be positioned over the threaded end 185 of the electrical plug 182. An electrical wire will extend from the electrical lug 184 to the voltage output 42. The electrical wire provides a path for voltage from the voltage output 42 to the electrical plug 182 which is then transferred to the probe electrode 100. A nut 186 may be used to secure the electrical plug 182 and the electrical lug 184 to the plastic insert 180. The closed end 142 of the probe electrode 100 is inserted in the receptacle end 183 of the electrical plug 182, as shown in FIG. 7.

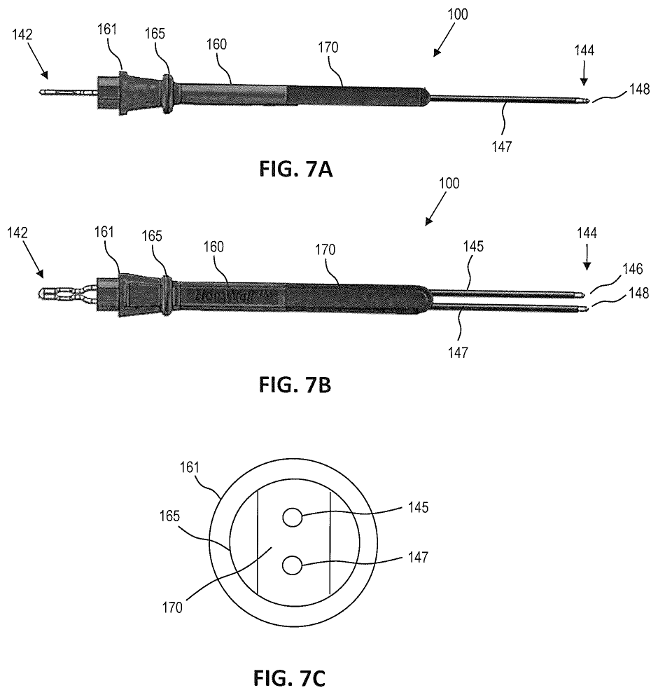

Referring now to FIGS. 8A-8C, a sequence of steps for inserting the closed end 142 of the probe electrode 100 into the receptacle end 183 of the electrical plug 182 will be discussed. As noted above, the electrical plug 182 is position within the opening in the plastic insert 180, and is secured to the plastic holder 180 using a nut 186 positioned on the threaded end 185 of the electrical plug 182

The closed end 142 of the probe electrode 100 is configured as a spring, as shown in FIG. 8A, for press-fitting into the receptacle end 183 of the electrical plug 182. As the tip 143 of the closed end 142 bottoms out within the receptacle end 183, the closed end 142 starts to expand outwards, as shown in FIG. 8B. With the closed end 142 fully inserted into the receptacle end 183 of the electrical plug 182, as shown in FIG. 8C, expansion of the closed end firmly holds the probe electrode 100 in place.

Although the plastic insert 180 is not shown, an outermost portion of the opening extending therethrough is shaped to receive the keying block 162 of the base overmold 160 of the probe electrode 100. Prior to insertion of the probe electrode 100, the keying block 162 may be rotated so that electrode tips 146, 148 are oriented in either a vertical or horizontal position with respect to how the electrode tips 146, 148 will be contacting the hemorrhoid 72.

Referring now to FIG. 9, the user interface membrane 190 positioned over the display area 53 of the handheld device 40 will be discussed. As noted above, the display 52 includes a current bar graph 54 and a timer 56. The user interface membrane 190 includes an opening for the current bar graph 54 and an opening for the timer 56.

The user interface membrane 190 is coupled to the controller 46 and provides user interface buttons for operating the handheld device 40. The user interface membrane 190 includes an on and up arrow button 192, an off and down arrow button 194, a self-test button 196 and a timer button 198.

A self-test of the handheld device 40 is to be run before each use, unless this function has been disabled by the doctor via software. To initiate the self-test, the doctor engages the grounding pad 80 with the probe electrode 100 and presses the self-test button 196.

During the self-test, each digit on the timer 56 is tested to make sure all the segments are working correctly. Then as part of the self-test, the handheld device 40 sweeps from delivering 0 to 16 milliamps (mA) and back to 0. As the current sweeps for 0 to 16 mA, the current bar graph 54 displays a bar at every 2 mA increment. When 16 mA is being delivered by the handheld device 40, then 8 bars appear. As the current sweeps back to 0, a bar will be removed every 2 mA decrement.

The self-test also requires the doctor to press the up arrow button 192 and the down arrow button 194 to make sure these buttons are working. If everything is working correctly, then “pass” is displayed on the timer 56. In order to remove display of “pass” the doctor presses the timer button 198. This causes all zeros to appear on the timer 56.

The doctor now has the option to operate the handheld device 40 in a manual mode or a timer mode. In the manual mode, the doctor engages the hemorrhoid 72 and then presses the up arrow button 192 for 3 seconds. The 3 second delay before current is delivered to the hemorrhoid 72 helps to prevent the handheld device 40 from accidentally delivering current when it is not intended to do so.

As current is being delivered to the hemorrhoid 72, the timer 56 counts up from zero. The handheld device 40 is configured to deliver 16 mA as a default current level setting. If the doctor wants less current to be delivered, then the doctor presses the down arrow button 194 until the desired number of bars appear on the timer 56, where each bar represents 2 mA.

If current is not being delivered to the hemorrhoid 72, then the timer 56 stops counting until current is actually being delivered again. For instance, the doctor may move the probe electrode 100 so that contact is no longer being made with the hemorrhoid 72. Once contact with the hemorrhoid 72 is again made by the probe electrode 100, then the timer 56 will display the bars indicating that current is being delivered by the handheld device 40. After an appropriate amount of time has passed in treating the hemorrhoid 72, the doctor presses the down arrow button 194.

For the doctor to operate the handheld device 40 in the timer mode, the doctor presses the timer button 198. This causes the timer 56 to flash or blink. Each press of the timer button 198 adds 30 seconds to the timer 56. After the desired amount of time has been entered, the doctor presses the timer button 56 again which causes the set time on the timer 56 to stop flashing. Next, the doctor presses the up arrow button 192 for 3 second. As current is being delivered to the hemorrhoid 72, the timer 56 counts down from the set time to zero.

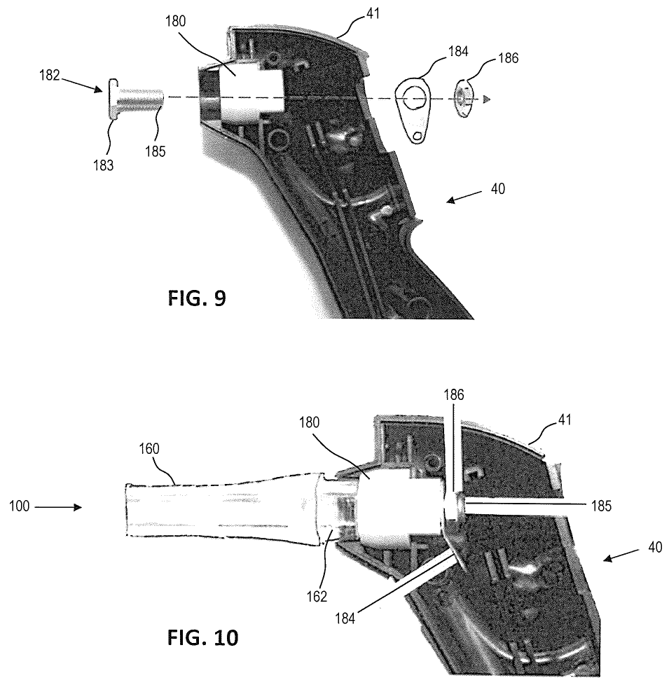

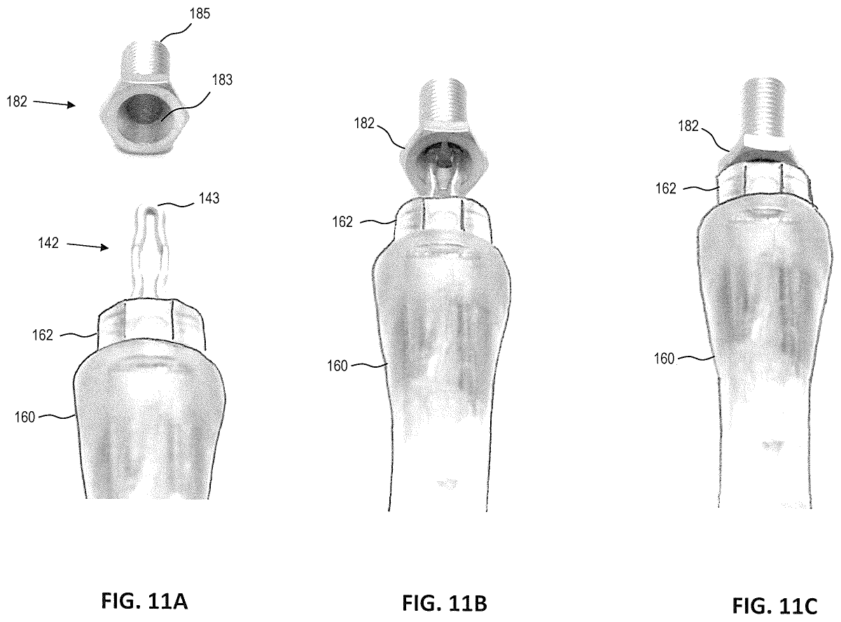

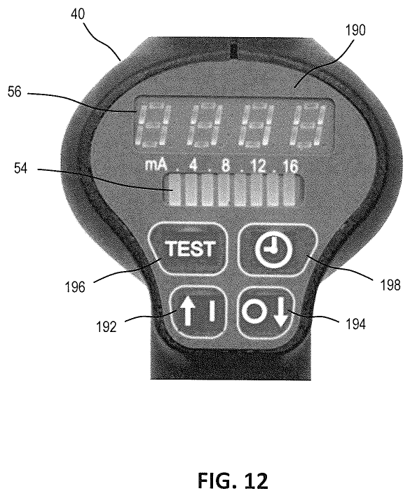

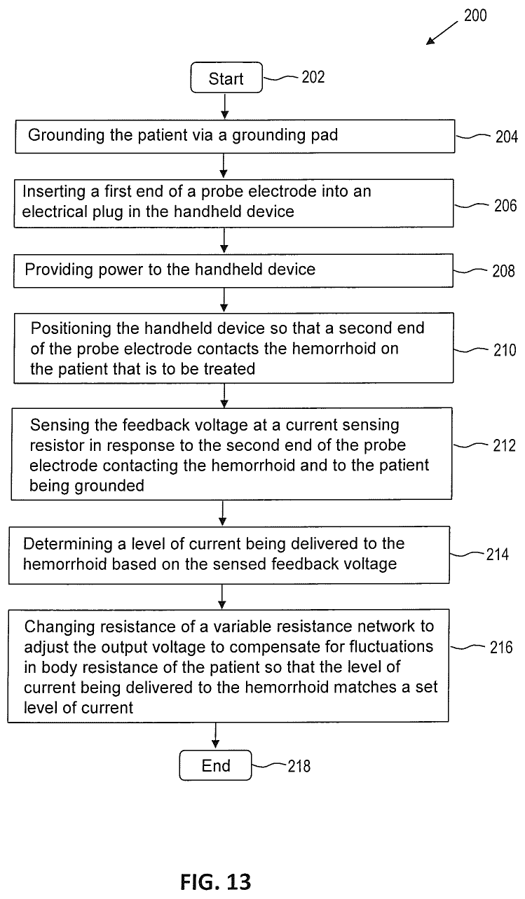

Another aspect is directed to a method for using the handheld device 40 as discussed above to treat a hemorrhoid 72 on a patient 70. Referring now to the flowchart 200 in FIG. 10, from the start (Block 202), the method includes grounding the patient 70 via the grounding pad 80 at Block 204. The first end of the probe electrode 100 is inserted into the electrical plug 182 at Block 206. Power is provided by the computing device 30 to the handheld device 40 over cable 60 at Block 208. The handheld device 40 is then positioned at Block 210 so that a second end of the probe electrode 100 contacts the hemorrhoid 72.

The controller 46 within the handheld device 40 is operated to sense the feedback voltage at the current sensing resistor 50 at Block 212 in response to the second end of the probe electrode 100 contacting the hemorrhoid 72 and to the patient 70 being grounded. A level of current being delivered to the hemorrhoid 72 based on the sensed feedback voltage is determined at Block 214.

The resistance of the variable resistance network 44 is changed at Block 216 to adjust the output voltage to compensate for fluctuations in body resistance of the patient 70 so that the level of current being delivered to the hemorrhoid matches a set level of current. The method ends at Block 218.

Many modifications and other embodiments will come to the mind of one skilled in the art having the benefit of the teachings presented in the foregoing descriptions and the associated drawings. Therefore, it is understood that the foregoing is not to be limited to the example embodiments, and that modifications and other embodiments are intended to be included within the scope of the appended claims.

1. A hemorrhoid treatment system comprising:
a computing device;
a handheld device comprising a cable coupled to the computing device, with the cable comprising an inline connector junction;
a probe electrode having a first end coupled to the handheld device, and a second end configured to contact a hemorrhoid on a patient that is to be treated;
a grounding pad configured to be coupled to the patient; and
a grounding pad cable coupled between the grounding pad and the inline connector junction;
the handheld device comprising:
a voltage output configured to deliver an output voltage to the probe electrode,
a variable resistance network coupled to the voltage output,
a current sensing resistor configured to provide a feedback voltage, and
a controller coupled to the current sensing resistor and the variable resistance network, and configured to perform the following:
sense the feedback voltage at the current sensing resistor in response to the second end of the probe electrode contacting the hemorrhoid and to the patient being grounded via the grounding pad,
determine a level of current being delivered to the hemorrhoid based on the sensed feedback voltage, and
change resistance of the variable resistance network to adjust the output voltage to compensate for fluctuations in body resistance of the patient so that the level of current being delivered to the hemorrhoid matches a set level of current.

2. The hemorrhoid treatment system according to claim 1 wherein the controller periodically senses the feedback voltage at the current sensing resistor.

3. The hemorrhoid treatment system according to claim 1 wherein the variable resistance network comprises a plurality of resistive inputs, with each resistive input corresponding to a different resistive value; and wherein the controller comprises a sensing and adjusting algorithm to periodically sense the feedback voltage, and to adjust the resistance of the variable resistance network by selecting one of the plurality of resistive inputs so that the level of current being delivered to the hemorrhoid matches the set level of current.

4. The hemorrhoid treatment system according to claim 1 wherein the cable is configured to provide power and ground from the computing device to the handheld device.

5. The hemorrhoid treatment system according to claim 4 wherein the grounding pad cable is coupled to ground in the inline connector junction.

6. The hemorrhoid treatment system according to claim 1 wherein the computing device comprises operational treatment software configured to perform handshaking with the handheld device in order for the handheld device to operate.

7. The hemorrhoid treatment system according to claim 1 wherein the computing device comprises operational treatment software configured to generate a treatment summary report in response to the handheld device being powered off after treatment of the hemorrhoid, with the treatment summary report to be stored in a medical records database.

8. The hemorrhoid treatment system according to claim 7 wherein the treatment summary report includes identification of the patient, a duration of how long current was delivered to the hemorrhoid, and the level of the current delivered to the hemorrhoid during the duration.

9. The hemorrhoid treatment system according to claim 1 wherein the handheld device comprises a timer display and is configured to operate in a manual mode or a timer mode, with the manual mode corresponding to the timer display counting up from zero during treatment of the hemorrhoid, and with the timer mode corresponding to the timer display counting down from a selected time set on the timer display.

10. The hemorrhoid treatment system according to claim 1 wherein the handheld device comprises a current bar graph display to display the current level being delivered to the hemorrhoid.

11. The hemorrhoid treatment system according to claim 1 wherein the probe electrode is bifurcated as a pair of spaced apart elongated electrode members each with an electrode tip for contacting the hemorrhoid.

12. The hemorrhoid treatment system according to claim 11 comprising a spacer for slidably engaging the elongated electrode members in order to maintain a desired spacing between the electrode tips.

13. The hemorrhoid treatment system according to claim 11 comprising a base overmold positioned over a portion of the elongated electrode members, and an open extension extending from the base between inward facing surfaces of the elongated electrode members while exposing outward facing surfaces of the elongated electrode members.

14. The hemorrhoid treatment system according to claim 1 wherein the probe electrode is a continuous length of conductive material folded in half to define a closed end and an open end, with the closed end configured as a spring for press-fitting into the handheld device, and with the open end configured as a pair of spaced apart electrode tips for contacting the hemorrhoid.

15. The hemorrhoid treatment system according to claim 1 wherein the handheld device comprises:
a housing enclosing the voltage output, the variable resistance network, the current sensing resistor and the controller;
a plastic insert secured within the housing and having an opening extending therethrough;
an electrical plug having a receptacle end and a threaded end, with the receptacle end positioned within the opening in the plastic insert and configured to receive the first end of the probe electrode, and with the threaded end extending through a backside of the plastic insert;
an electrical lug positioned over the threaded end of the electrical plug for contacting the voltage output; and
a nut secured to the threaded end of the electrical plug for securing the electrical plug within the plastic insert.

16. The hemorrhoid treatment system according to claim 1 wherein the handheld device comprises at least one light directed to the second end of the probe electrode that contacts the hemorrhoid.

17. A handheld device comprising:
a housing;
an electrical plug carried by the housing and having a receptacle end configured to receive a first end of a probe electrode, with a second end of the probe electrode configured to contact a hemorrhoid on a patient that is to be treated;
a voltage output carried by the housing and configured to deliver an output voltage to the probe electrode via the electrical plug;
a variable resistance network carried by the housing and coupled to the voltage output;
a current sensing resistor carried by the housing and configured to provide a feedback voltage; and
a controller carried by the housing and coupled to the current sensing resistor and the variable resistance network, and configured to perform the following:
sense the feedback voltage at the current sensing resistor in response to the second end of the probe electrode contacting the hemorrhoid and to the patient being grounded,
determine a level of current being delivered to the hemorrhoid based on the sensed feedback voltage, and
change resistance of the variable resistance network to adjust the output voltage to compensate for fluctuations in body resistance of the patient so that the level of current being delivered to the hemorrhoid matches a set level of current.

18. The handheld device according to claim 16 wherein the controller periodically senses the feedback voltage at the current sensing resistor.

19. The handheld device according to claim 16 wherein the variable resistance network comprises a plurality of resistive inputs, with each resistive input corresponding to a different resistive value; and wherein the controller comprises a sensing and adjusting algorithm to periodically sense the feedback voltage, and to adjust the resistance of the variable resistance network by selecting one of the plurality of resistive inputs so that the level of current being delivered to the hemorrhoid matches the set level of current.

20. The handheld device according to claim 16 comprising a cable to be coupled to a computing device, with the computing device to provide power and ground to the handheld device, and with the cable comprising an inline connector junction that is to interface with a grounding pad cable coupled to a grounding pad for grounding the patient

21. The handheld device according to claim 16 comprises a timer display, with the handheld device configured to operate in a manual mode or a timer mode, with the manual mode corresponding to the timer display counting up from zero during treatment of the hemorrhoid, and with the timer mode corresponding to the timer display counting down from a selected time set on the timer display.

22. The handheld device according to claim 16 comprises a current bar graph display to display the current level being delivered to the hemorrhoid by the handheld device.

23. The handheld device according to claim 16 wherein the handheld device comprises:
a housing enclosing the voltage output, the variable resistance network, the current sensing resistor and the controller;
a plastic insert secured within the housing and having an opening extending therethrough;
an electrical plug having a receptacle end and a threaded end, with the receptacle end positioned within the opening in the plastic insert and configured to receive the first end of the probe electrode, and with the threaded end extending through a backside of the plastic insert;
an electrical lug positioned over the threaded end of the electrical plug for contacting the voltage output; and
a nut secured to the threaded end of the electrical plug for securing the electrical plug within the plastic insert.

24. The handheld device according to claim 16 wherein the handheld device comprises at least one light directed to the second end of the probe electrode that contacts the hemorrhoid.

25. A method for using a handheld device to treat a hemorrhoid on a patient, the handheld device comprising a housing; an electrical plug carried by the housing and having a receptacle end configured to receive a first end of a probe electrode; a voltage output carried by the housing to deliver an output voltage to the probe electrode via the electrical plug; a variable resistance network carried by the housing and coupled to the voltage output; a current sensing resistor carried by the housing to provide a feedback voltage; and a controller carried by the housing and coupled to the current sensing resistor and the variable resistance network, the method comprising:
grounding the patient via a grounding pad;
providing power to the handheld device;
positioning the handheld device so that a second end of the probe electrode contacts the hemorrhoid on the patient; and
operating the controller to perform the following:
sense the feedback voltage at the current sensing resistor in response to the second end of the probe electrode contacting a hemorrhoid on a patient that is to be treated and to the patient being grounded,
determine a level of current being delivered to the hemorrhoid based on the sensed feedback voltage, and
change resistance of the variable resistance network to adjust the output voltage to compensate for fluctuations in body resistance of the patient so that the level of current being delivered to the hemorrhoid matches a set level of current.
